(19) United States
(12) Patent Application Publication
(10) Pub. No.: US 2007/0113951 A1
(43) Pub. Date: May 24, 2007

(54) Osteochondral composite scaffold for articular cartilage repair and preparation thereof

(75) Inventor: Ta-Jen Huang, Hsinchu (TW)

Correspondence Address: BACON & THOMAS, PLLC, 625 SLATERS LANE, FOURTH FLOOR, ALEXANDRIA, VA, 22314, US

(73) Assignee: National Tsing Hua University, Hsinchu (TW)

(21) Appl. No.: 11/304,707

(22) Filed: Dec. 16, 2005

(30) Foreign Application Priority Data
Nov. 24, 2005 (TW) 94139017

(51) Int. Cl. A61F 2/28

(52) U.S. Cl. 156/89.11; 623/14.12; 623/23.51; 623/23.56; 427/2.27; 427/372.2

(57) ABSTRACT

The present invention discloses a biomedical scaffold material for articular cartilage repair, which is a multi-layer composite scaffold in the cylindrical plug form. It includes a lower porous ceramic layer intimating the bone zone of the joint, and an upper porous ceramic layer intimating the bottom cartilage zone of the joint; a dense ceramic separation layer connecting the lower and upper porous ceramic layers; and a porous gelatin layer, intimating the middle cartilage zone of the joint, affixed to the upper porous ceramic layer.

### FIELD OF THE INVENTION

The present invention relates to an osteochondral composite scaffold for articular cartilage repair, particularly a composite scaffold in a cylindrical plug form for articular cartilage repair.

### BACKGROUND OF THE INVENTION

Osteoarthritis not only will cause wearing of articular cartilage, but also, when in its severe state, will cause the blood vessels of the bone under the articular cartilage penetrating through the calcified layer and into the cartilage zone, and cause an excessive growth of the bone, thereby forming spur and completely sabotaging the functions of the articular cartilage. Generally, when a tissue engineering scaffold is implanted into the joint of a patient suffering from osteoarthritis, the damages on the articular cartilage will reoccur in a short term due to excessive growth of the bone even the damages are fully repaired initially, because the penetration of the blood vessels from the bone under the articular cartilage can not be stopped. Therefore, the recurrence of osteoarthritis can be avoided only if the damaged cartilage and the calcified region, together with the bone underneath, are replaced with a tissue engineering scaffold with a separation layer.

### SUMMARY OF THE INVENTION

One objective of the present invention is to provide a tissue engineering scaffold to be applied on articular cartilage repair.

The present invention provides an osteochondral composite scaffold simulating an articular joint for articular cartilage repair, wherein the composite scaffold can promote in vitro culture of articular chondrocytes.

An osteochondral composite scaffold for the repair of articular cartilage constructed according to the present invention includes a dense layer for separating the cartilage zone from the bone zone (i.e. a separation layer) in order to achieve the effect of preventing blood vessels from penetrating into the cartilage zone from the bone zone.

Preferred embodiments of the present invention include (but not limited to) the following:

1. An osteochondral composite scaffold for articular cartilage repair, which comprises:

a lower porous ceramic layer intimating the bone zone of an articular joint;

an upper porous ceramic layer intimating the bottom cartilage zone of the joint; and

a dense ceramic separation layer connecting the lower porous ceramic layer to the upper porous ceramic layer; and

optionally a porous bio-polymer matrix layer affixed to the upper porous ceramic layer, intimating the middle cartilage zone of the joint.

2. The composite scaffold as recited in Item 1, wherein the separation layer is a hardened or sintered calcium phosphate cement, calcium sulfate cement, or bioglass, with a pore size less than 5 μm.

3. The composite scaffold as recited in Item 2, wherein the separation layer is a hardened or sintered calcium phosphate cement.

4. The composite scaffold as recited in Item 3, wherein the calcium phosphate cement comprises tricalcium phosphate powder.

5. The composite scaffold as recited in Item 2, wherein the separation layer has a thickness less than 1 mm.

6. The composite scaffold as recited in Item 1, which comprises the porous bio-polymer matrix layer.

7. The composite scaffold as recited in Item 6, wherein the porous bio-polymer matrix layer is gelatin or collagen.

8. The composite scaffold as recited in Item 7, wherein the gelatin or collagen is a cross-linked gelatin or collagen by a cross-linking agent.

9. The composite scaffold as recited in Item 6, wherein the porous bio-polymer matrix layer has a porosity of 90-95 vol % and a pore size of 200-500 μm.

10. The composite scaffold as recited in Item 6, wherein the porous bio-polymer matrix layer has a thickness of 1-3 mm.

11. The composite scaffold as recited in Item 1, wherein the lower porous ceramic layer is a hardened or sintered calcium phosphate cement, calcium sulfate cement, or bioglass, with a porosity of 20-30 vol % and a pore size of 100-200 μm.

12. The composite scaffold as recited in Item 11, wherein the lower porous ceramic layer is a sintered calcium phosphate cement.

13. The composite scaffold as recited in Item 12, wherein the calcium phosphate cement comprises calcium polyphosphate powder.

14. The composite scaffold as recited in Item 11, wherein the lower porous ceramic layer has a thickness of 2-5 mm.

15. The composite scaffold as recited in Item 1, wherein the upper porous ceramic layer is a hardened or sintered calcium phosphate cement, calcium sulfate cement, or bioglass, with a porosity of 10-50 vol % and a pore size of 50-300 μm.

16. The composite scaffold as recited in Item 15, wherein the upper porous ceramic layer is a sintered calcium phosphate cement.

17. The composite scaffold as recited in Item 16, wherein the calcium phosphate cement comprises calcium polyphosphate powder.

18. The composite scaffold as recited in Item 15, wherein the upper porous ceramic layer has a thickness of 0.2-2 mm.

19. The composite scaffold as recited in Item 1, which is a cylinder with a diameter of 5-20 mm.

20. The composite scaffold as recited in Item 6, which further comprises chondrocytes adhered to and tissues grown in the porous bio-polymer matrix layer.

21. A method for preparing an osteochondral composite scaffold for articular cartilage repair, which comprises:

a) compressing a first porous ceramic precursor powder to form a lower porous ceramic layer green body;

b) disposing a dense ceramic separation layer on a surface of the lower porous ceramic layer green body; or coating a layer of a paste formed of a dense ceramic precursor powder and an aqueous solution on the surface of the green body, and hardening the paste on the surface to form a dense ceramic separation layer;

c) disposing a hollow columnar mold on the separation layer, and pouring a second porous ceramic precursor powder into the mold to stack the second porous ceramic precursor powder on the separation layer; or compressing a second porous ceramic precursor powder to form an upper porous ceramic layer green body, and disposing the green body on the separation layer; and

d) sintering the resulting stacked structure from step c) to form a sandwiched structure formed of an upper porous ceramic layer, a separation layer, and a lower porous ceramic layer.

22. The method as recited in Item 21, which further comprises:

e) preparing a bio-polymer solution;

f) disposing a hollow columnar mold on the upper porous ceramic layer of the sandwiched structure, pouring the bio-polymer solution into the mold to form a reservoir of the bio-polymer solution, cooling the reservoir to form a gel-like material and then removing the mold;

g) contacting the gel-like material with an aqueous solution containing a cross-linking agent to form a cross-linked bio-polymer block; and

h) washing the cross-linked bio-polymer block, and freeze-drying the washed block to form a porous bio-polymer matrix layer affixed to the upper porous ceramic layer.

23. The method as recited in Item 21, which further comprises:

e′) preparing an aqueous solution containing a bio-polymer and a cross-linking agent;

f′) disposing a hollow columnar mold on the upper porous ceramic layer of the sandwiched structure, pouring the aqueous solution into the mold to form a reservoir, cooling the reservoir to form a gel-like material and then removing the mold;

g′) aging the gel-like material to form a cross-linked bio-polymer block; and

h) washing the cross-linked bio-polymer block, and freeze-drying the washed block to form a porous bio-polymer matrix layer affixed to the upper porous ceramic layer.

24. The method as recited in Item 22, which further comprises:

i) wetting the porous bio-polymer matrix layer, and then freeze-drying the matrix layer to form a porous bio-polymer matrix layer with a different structure.

25. The method as recited in Item 23, which further comprises: i) wetting the porous bio-polymer matrix layer, and then freeze-drying the matrix layer to form a porous bio-polymer matrix layer with a different

### BRIEF DESCRIPTION OF THE DRAWINGS

FIG. 1 shows a schematic cross-sectional view of an osteochondral composite scaffold for articular cartilage repair according to a preferred embodiment of the present invention;

FIG. 2A shows a Scanning Electron Microscopy (SEM) photo of a porous gelatin matrix formed by cooling an aqueous solution containing 5 wt % of gelatin, cross-linking by using an aqueous solution containing 0.5 wt % of glutaraldehyde (GA), and freeze-drying once;

FIG. 2B shows a SEM photo of a porous gelatin matrix formed by cooling an aqueous solution containing 5 wt % of gelatin, cross-linking by using an aqueous solution containing 0.5 wt % of genipin (GP), and freeze-drying once;

FIG. 2C shows a SEM photo of a porous gelatin matrix formed by cooling an aqueous solution containing 5 wt % of gelatin, cross-linking by using an aqueous solution containing 0.5 wt % of GA, and freeze-drying twice;

FIG. 2D shows a SEM photo of a porous gelatin matrix formed by cooling an aqueous solution containing 5 wt % of gelatin, cross-linking by using an aqueous solution containing 0.5 wt % of GP, and freeze-drying twice;

FIG. 3A shows a magnified photograph taken by an optical microscope of a tissue slice taken from a porous gelatin matrix after being embedded in paraffin and stained with hematoxylin-eosin, which is prepared by GP-cross-linking, followed by freeze-drying twice, implanting with 5×10⁶ cells and culturing for 30 days; and

FIG. 3B is a further magnified photograph of a portion of the tissue slice shown in FIG. 3A taken by an optical microscope.

### DETAILED DESCRIPTION OF THE INVENTION

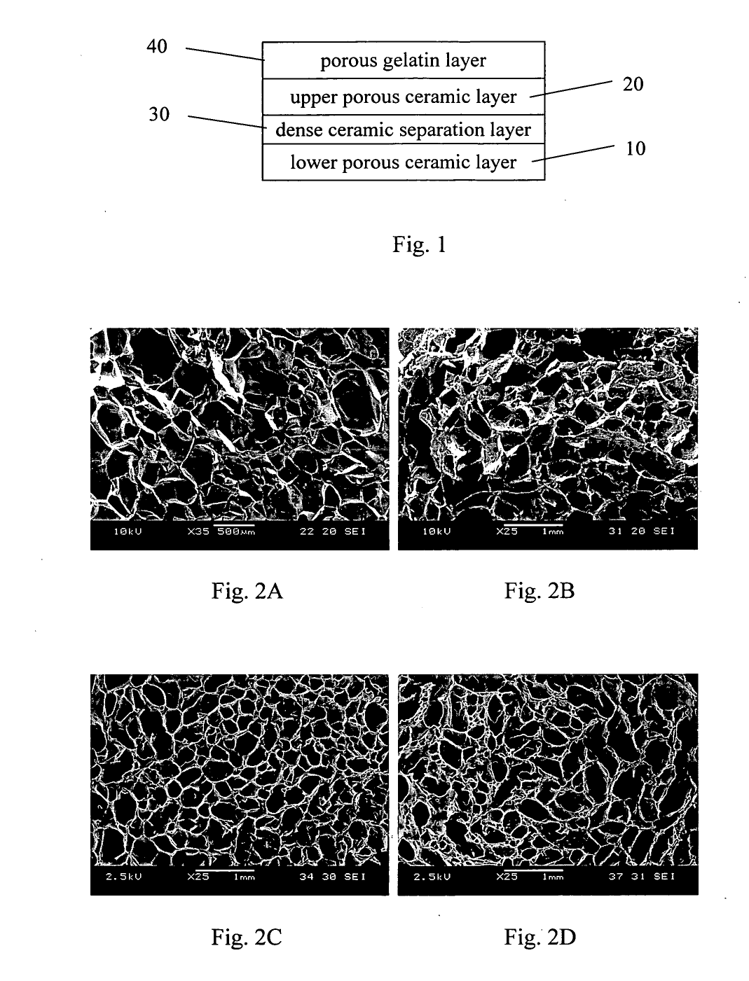

As shown in FIG. 1, an osteochondral composite scaffold for articular cartilage repair according to a preferred embodiment of the present invention includes:

a lower porous ceramic layer 10 intimating the bone zone of the articular joint;

an upper porous ceramic layer 20 intimating the bottom cartilage zone of the articular joint;

a dense ceramic separation layer 30 connecting the lower porous ceramic layer to the upper porous ceramic layer; and

a porous gelatin layer 40, intimating the middle cartilage zone of the articular joint, affixed to the upper porous ceramic layer.

In the present invention, for the purpose of accelerating the rate of in vitro culture of articular chondrocytes, a porous gelatin layer 40 for accelerating the growth of cartilage tissues is affixed to the porous ceramic layer 20. In addition to gelatin, which is a biological polymer, any polymer material capable of accelerating the rate of in vitro chondrocyte culture can also be used.

The function of each layer in the composite scaffold of the present invention is described as follows:

(1) The lower porous ceramic layer 10 intimating the bone zone of the articular joint: intimating subchondral bone, cancellous bone, and cortical bone. The material for the bone zone is selected from calcium phosphate, which is a biomedical ceramic material, e.g. β-calcium polyphosphate (β-CPP), with a thickness of 3 mm, a porosity of 20˜30 vol %, and a pore size of about 100˜200 μm.

(2) The upper porous ceramic layer 20 intimating the bottom cartilage zone of the articular joint: intimating the calcified zone of the articular cartilage. The material intimating the calcified zone of the articular cartilage (cartilage bottom layer) is selected from calcium phosphate, β-CPP, with a thickness of 0.2˜2 mm, a porosity of 10˜50 vol %, a pore size of 50˜300 μm, which may vary depending on whether the porous gelatin layer 40 is provided.

(3) The porous gelatin layer 40 intimating the middle cartilage zone of the articular joint: the matrix of the layer 40 has a thickness of 2 mm, a porosity of 90˜95 vol %, and a pore size of 200˜500 μm. The porous gelatin layer 40 can be made from pigskin gelatin. The gelatin is a denatured product of collagen and contains a RGD sequence capable of assisting the adhesion and growth of chondrocytes, as well as maintaining the cell activities. However, an un-processed gelatin is easy to degrade, and a gelatin will absorb water, becoming soft and lack of sufficient anti-compression mechanical strength. Thus, preferably, gelatin is cross-linked by a cross-linking agent, e.g. glutaraldehyde (GA) or genipin (GP), to enhance the thermal stability and anti-compression strength of the structure of the porous gelatin layer 40.

(4) Separation layer 30: a thin layer separating the bone zone from the cartilage zone. The material for the separation layer is selected from calcium phosphate, e.g. β-tricalcium phosphate (β-TCP). The separation layer is the thinner the better, wherein the separation layer needs to have a porosity <5 vol % and a pore size <5 μm.

Experiments

### Process for Producing Composite Scaffold

1. Preparation of Composite Scaffold (wherein the processes for preparing the separation layer and the porous gelatin layer will be described following this section)

(1) 0.3 g of amorphous calcium polyphosphate (aCPP) powder was compressed at 5 tons of pressure to form a green body 10 mm in diameter (this aCPP layer is used to intimate the bone layer).
(2) A thin disc of β-TCP separation layer was placed on the green body or a thin β-TCP separation layer was formed on the green body by coating a paste of β-TCP powder. Next, a hollow cylindrical mold was disposed on the separation layer. 0.04 g of aCPP powder was poured into the mold in order to form a cylinder or a disc of aCPP powder on the green body. In the situation where a porous gelatin layer is used to accelerate the growth of cartilage tissues, the cylinder or disc of aCPP powder can be formed by a simpler compression process, so that a compressed aCPP green body can be stacked on the separation layer, wherein the compression process may be similar to that in step (1) or a like process for producing thin disc.
(3) Temperature was raised to 900° C. at 10° C./min and kept at 900° C. for 2 hr, and then annealed in air.
(4) The mold was removed and then the ceramic structure was washed with deionized water, immersed in absolute alcohol, and dried in an oven at 110° C., thereby producing a semi-product with a sandwich structure, which was stored in a desiccator. The thickness of each layer is: 1 mm for the upper porous ceramic layer; 0.61 mm for the separation layer; and 3 mm for the lower porous ceramic layer.
   2. Preparation of the Separation Layer
(1) 95 g of β-TCP and 5 g of Na₄P₂O₇.10H₂O (sodium pyrophosphate) were added in 100 mL of deionized water, and stirred thoroughly.
(2) Water was removed from the mixture in an oven at 90° C.
(3) The resulting solid was pulverized in a pulverizer and the resulting powder was stored in a desiccator.
(4) 0.1 g of powder was compressed at 4 ton of pressure to form a green body of disc 10 mm in diameter.
(5) The green body was heated to 1180° C. at 5° C./min and kept at that temperature for 6 hr and then annealed in air.
(6) The ceramic disc was washed with deionized water, immersed in absolute alcohol, dried in an oven at 110° C., and then stored in a desiccator.
   3. Preparation of the Porous Gelatin Layer

A process for preparing the porous gelatin layer comprised converting a gelatin at a low temperature into a jelly-like gel; immersing the jelly-like gel in a solution of a cross-linking agent to undergo a cross-linking reaction; upon completion of the reaction, performing washing, freezing, and freeze-drying steps.

Experimental Steps:

(1) Preparing various gelatin aqueous solutions at different wt %; which were heated in a thermostat bath at 50° C. for 1 hr under agitation;
(2) Disposing a hollow cylindrical mold on the upper porous ceramic layer of the semi-product with a sandwiched structure prepared in the above 1; pouring the solution from step (1) into the mold, and placing the resulting semi-product with the mold in a refrigerator to cool the solution into a gel; and then removing the mold, wherein the gelatin solution had migrated into the upper porous ceramic layer and the gelatin layer had been affixed to the upper porous ceramic layer after gel formation;
(3) Immersing the composite scaffold in a solution containing 0.5 wt % of glutaraldehyde (GA) or 0.5 wt % of genipin (GP) to cross-link the gel at room temperature for two days;
(4) Removing the composite scaffold from the solution and washing it with aniline, followed by washing it with deionized water three times;
(5) Freezing the composite scaffold in a freezer at 20° C. for 3 hrs;
(6) Freeze-drying the composite scaffold in a vacuum freeze-dryer (−55° C. and 100 mtorr) for 36 hrs; and
(7) Rinsing the composite scaffold at room temperature, and then performing another freeze-drying on the composite scaffold.

This process can be used to produce a single porous gelatin matrix, used as an individual scaffold, which only requires pouring the solution of step (1) into a cylindrical container (mold) as in step (2). The rest of the steps remained the same.

In the present invention, the process of affixing the porous gelatin layer to the porous ceramic layer can also adopt the following simple step (2′) before the steps (4)˜(7), i.e. replacing the steps (2)˜(3) with:
(2′) Mixing the solution of step (1) with 0.5 wt % of GA or 0.5 wt % of GP; stirring the resulting solution in a thermostat bath at 50° C. for 2 minutes; pouring the solution into the mold used in the above step (2); cross-linking the solution at room temperature for two days and removing the mold.

### Evaluation of Composite Scaffold

1. Evaluation of the Separation Layer

The separation layer is a thin layer separating the cartilage zone from the bone zone with a function of stopping the blood vessels in the bone zone to penetrate into the cartilage zone. The material of the separation layer was β-TCP. The separation layer was the thinner the better (where the thickness of the separation layer can be very small if a coating process is used). The separation layer needs to have a porosity of <5 vol %, and a pore size <5 μm.

According to the experimental results, the sintered β-TCP separation layer had a diameter of 8.36 mm and a thickness of 0.61 mm. The porosity of the TCP separation layer was reduced from 46 vol % before sintering to 3 vol % after sintering. A Scanning Electron Microscopy (SEM) photograph shows that the TCP separation layer has almost no pores. Thus, the β-TCP ceramic film is a suitable separation layer material.

2. Evaluation of Composite Scaffold (Evaluation of Porous Gelain Layer will be Described Following this Section) —Effect on Chondrocyte Culture

The content of the glycosaminoglycan (GAG) in the extracellular matrix of the cartilage tissues grown in the tissue engineering scaffold should be 3˜5 times of the content of hydroxyproline (HP) so as to conform to the composition of extracellular matrix of natural cartilage tissues. The results of this experiment complied with this requirement quite well.

After about one month of in vitro culture, the slice of the composite scaffold of this experiment stained by toluidine blue are similar to that of the slice of a natural cartilage.

3. Evaluation of the Porous Gelatin Layer

After the porous gelatin layer was affixed to the upper porous ceramic layer, an experiment was carried out by shaking the composite scaffold in an aqueous solution simulating the in vitro culture of cartilage tissue. The experimental results show that the adhesion between the porous gelatin layer and the upper porous ceramic layer is strong and the porous gelatin layer does not delaminate from the upper porous ceramic layer. This adhesion can be enhanced by adjusting parameters such as the porosity and thickness of the upper porous ceramic layer. The following text will describe the properties of a single porous gelatin matrix, used as an individual scaffold, and their influences on chondrocyte culture in order to identify the factors of the process for preparing the porous gelatin layer of the present invention.

An experiment was carried out to observe the performance of a porous gelatin matrix cross-linked at 25° C.: The results indicate that the porous gelatin matrix show no dissolution or disintegration. The SEM observations on the GA- and GP- cross-linked porous gelatin matrixes show that the pore sizes of the matrixes substantially are within 300˜500 μm for those being subjected to the freeze-drying treatment once or twice. The walls of the porous gelatin matrix receiving the freeze-drying treatment twice are conspicuously different from the walls of the porous gelatin scaffold receiving the freeze-drying treatment once. As shown in FIG. 2A to 2D, the porous gelatin matrix receiving the freeze-drying treatment twice has a reduced pore size and increased number of pores, and is more uniform in structure. Furthermore, the walls of such pores have many small voids. Such a structure not only will increase the mechanical strength of the matrix, but also will assist the growth of tissue cells resulting from a faster migration of the cells and a better transfer of the culture medium. Influence of the Porous Gelatin matrix on Chondrocyte Culture A porous gelatin matrix obtained by GA or GP cross-linking and freeze-drying twice was implanted with 5×10⁶ cells. After nine days of culture, the porous gelatin matrix was embedded in paraffin and subjected to a tissue slice staining analysis. The results clearly indicate that the chondrocytes can adhere to the GA-or GP-cross-linked porous gelatin matrix. The GA-cross-linked porous gelatin matrix, due to the toxicity of GA, has fewer chondrocytes adhered to the scaffold and is unable to grow a tissue similar to the natural cartilage tissue. The GP-cross-linked porous gelatin matrix has many chondrocytes adhered thereto, and the density and the pattern of the cells are similar to those shown on the slice of the articular cartilage of a Wistar rat. In order to understand whether the chondrocytes can uniformly distribute inside a porous gelatin matrix, the slices of cross-sections and longitudinal sections of the GP-cross-linked porous gelatin matrix were used to observe the distribution of chondrocytes after a nine-day culture. The results indicate that the chondrocytes are uniformly distributed in the pores of the porous gelatin matrix and deep inside the porous gelatin matrix. The above observations verify that the GP-cross-linked porous gelatin matrix is suitable for chondrocyte growth.

Other than GP and GA, the present invention can also adopt other cross-linking agents to carry out cross-linking of the gelatin in order to form a porous gelatin layer or gelatin matrix with a sufficient mechanical strength.

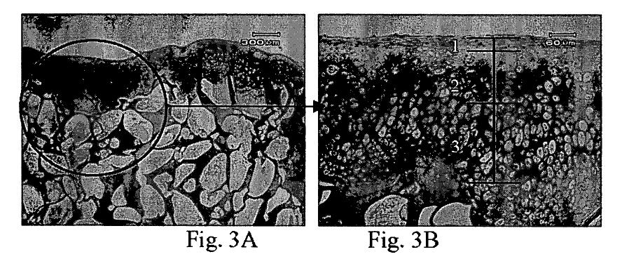

After 30 days of culture, the appearance of the porous gelatin matrix (as shown in FIG. 3A) shows that a cartilage tissue over-layer has developed. It can be seen from FIG. 3A that the thickness of the layer of cartilage tissues over the surface of the matrix is about 300 μm. As shown in FIG. 3B, the cartilage tissue developed is similar to the natural articular cartilage tissue, wherein the tissue layer marked as 1 is a superficial zone, the tissue layer marked as 2 is a middle zone, and the tissue layer marked as 3 is a deep zone. The above results show that the gelatin matrix of the invention is very suitable for the culture of the cartilage tissue.

4. Conclusion of Evaluation:

In view of above it can be concluded that the composite scaffold of the invention is a biomedical scaffold material suitable for articular cartilage repair.

In another embodiment of the invention, a sandwiched scaffold without the porous gelatin layer was implanted with chondrocytes for cell culture experiment. The experimental results indicate that the sandwiched scaffold free of the porous gelatin layer can also grow a cartilage tissue similar to the natural articular cartilage tissue (with a slower growth rate). Thus, a sandwiched scaffold without a porous gelatin layer can also be used as a biomedical scaffold material suitable for articular cartilage repair.

1. An osteochondral composite scaffold for articular cartilage repair, which comprises:
a lower porous ceramic layer intimating the bone zone of an articular joint;
an upper porous ceramic layer intimating the bottom cartilage zone of the joint; and
a dense ceramic separation layer connecting the lower porous ceramic layer to the upper porous ceramic layer; and
optionally a porous bio-polymer matrix layer affixed to the upper porous ceramic layer, intimating the middle cartilage zone of the joint.

2. The composite scaffold as claimed in claim 1, wherein the separation layer is a hardened or sintered calcium phosphate cement, calcium sulfate cement, or bioglass, with a pore size less than 5 μm.

3. The composite scaffold as claimed in claim 2, wherein the separation layer is a hardened or sintered calcium phosphate cement.

4. The composite scaffold as claimed in claim 3, wherein the calcium phosphate cement comprises tricalcium phosphate powder.

5. The composite scaffold as claimed in claim 2, wherein the separation layer has a thickness less than 1 mm.

6. The composite scaffold as claimed in claim 1, which comprises the porous bio-polymer-matrix layer.

7. The composite scaffold as claimed in claim 6, wherein the porous bio-polymer matrix layer is gelatin or collagen.

8. The composite scaffold as claimed in claim 7, wherein the gelatin or collagen is a cross-linked gelatin or collagen by a cross-linking agent.

9. The composite scaffold as claimed in claim 6, wherein the porous bio-polymer matrix layer has a porosity of 90-95 vol % and a pore size of 200-500 μm.

10. The composite scaffold as claimed in claim 6, wherein the porous bio-polymer matrix layer has a thickness of 1-3 mm.

11. The composite scaffold as claimed in claim 1, wherein the lower porous ceramic layer is a hardened or sintered calcium phosphate cement, calcium sulfate cement, or bioglass, with a porosity of 20-30 vol % and a pore size of 100-200 μm.

12. The composite scaffold as claimed in claim 11, wherein the lower porous ceramic layer is a sintered calcium phosphate cement.

13. The composite scaffold as claimed in claim 12, wherein the calcium phosphate cement comprises calcium polyphosphate powder.

14. The composite scaffold as claimed in claim 11, wherein the lower porous ceramic layer has a thickness of 2-5 mm.

15. The composite scaffold as claimed in claim 1, wherein the upper porous ceramic layer is a hardened or sintered calcium phosphate cement, calcium sulfate cement, or bioglass, with a porosity of 10-50 vol % and a pore size of 50-300 μm.

16. The composite scaffold as claimed in claim 15, wherein the upper porous ceramic layer is a sintered calcium phosphate cement.

17. The composite scaffold as claimed in claim 16, wherein the calcium phosphate cement comprises calcium polyphosphate powder.

18. The composite scaffold as claimed in claim 15, wherein the upper porous ceramic layer has a thickness of 0.2-2 mm.

19. The composite scaffold as claimed in claim 1, which is a cylinder with a diameter of 5-20 mm.

20. The composite scaffold as claimed in claim 6, which further comprises chondrocytes adhered to and tissues grown in the porous bio-polymer matrix layer.

21. A method for preparing an osteochondral composite scaffold for articular cartilage repair, which comprises:
a) compressing a first porous ceramic precursor powder to form a lower porous ceramic layer green body;
b) disposing a dense ceramic separation layer on a surface of the lower porous ceramic layer green body; or coating a layer of a paste formed of a dense ceramic precursor powder and an aqueous solution on the surface of the green body, and hardening the paste on the surface to form a dense ceramic separation layer;
c) disposing a hollow columnar mold on the separation layer, and pouring a second porous ceramic precursor powder into the mold to stack the second porous ceramic precursor powder on the separation layer; or compressing a second porous ceramic precursor powder to form an upper porous ceramic layer green body, and disposing the green body on the separation layer; and
d) sintering the resulting stacked structure from step c) to form a sandwiched structure formed of an upper porous ceramic layer, a separation layer, and a lower porous ceramic layer.

22. The method as claimed in claim 21, which further comprises:
e) preparing a bio-polymer solution;
f) disposing a hollow columnar mold on the upper porous ceramic layer of the sandwiched structure, pouring the bio-polymer solution into the mold to form a reservoir of the bio-polymer solution, cooling the reservoir to form a gel-like material and then removing the mold;
g) contacting the gel-like material with an aqueous solution containing a cross-linking agent to form a cross-linked bio-polymer block; and
h) washing the cross-linked bio-polymer block, and freeze-drying the washed block to form a porous bio-polymer matrix layer affixed to the upper porous ceramic layer.

23. The method as claimed in claim 21, which further comprises:
e′) preparing an aqueous solution containing a bio-polymer and a cross-linking agent;
f′) disposing a hollow columnar mold on the upper porous ceramic layer of the sandwiched structure, pouring the aqueous solution into the mold to form a reservoir, cooling the reservoir to form a gel-like material and then-removing the mold;
g′) aging the gel-like material to form a cross-linked bio-polymer block; and
h) washing the cross-linked bio-polymer block, and freeze-drying the washed block to form a porous bio-polymer matrix layer affixed to the upper porous ceramic layer.

24. The method as claimed in claim 22, which further comprises:
i) wetting the porous bio-polymer matrix layer, and then freeze-drying the matrix layer to form a porous bio-polymer matrix layer with a different structure.

25. The method as claimed in claim 23, which further comprises:
i) wetting the porous bio-polymer matrix layer, and then freeze-drying the matrix layer to form a porous bio-polymer matrix layer with a different structure.
